(19) United States
(12) Patent Application Publication
(10) Pub. No.: US 2023/0157629 A1
(43) Pub. Date: May 25, 2023

(54) SLEEP ONSET DETERMINATION METHOD, SLEEP ONSET DETERMINATION SYSTEM, AIR CONDITIONING DEVICE, AND RECORDING MEDIUM

(71) Applicant: Panasonic Intellectual Property Management Co., Ltd., Osaka (JP)

(72) Inventors: Takayuki NISHIKAWA, Osaka (JP); Seiya HIGUCHI, Osaka (JP); Yu SAKATA, Shiga (JP); Masahiro TANIKAWA, Osaka (JP); Kyohei KOIZUMI, Hyogo (JP)

(21) Appl. No.: 17/632,630

(22) PCT Filed: Jun. 11, 2021
(86) PCT No.: PCT/JP2021/022260

§ 371 Date: Feb. 3, 2022

(30) Foreign Application Priority Data
Feb. 19, 2021 (JP) 2021-025687

(51) Int. Cl. A61B 5/00; F24F 11/66

(52) U.S. Cl. CPC A61B 5/4809; F24F 11/66; F24F 2120/12

(57) ABSTRACT

A sleep onset determination method includes: detecting a light-out time that is a time at which illuminance in a room becomes less than or equal to a predetermined value; storing the light-out time detected in the detecting of the light-out time and a time period including the light-out time detected; and determining whether a user fell asleep at a currently detected light-out time, based on the light-out time and the time period stored in the storing of the light-out time.

### CROSS-REFERENCE OF RELATED APPLICATIONS

This application is the U.S. National Phase under 35 U.S.C. § 371 of International Patent Application No. PCT/JP2021/022260, filed on Jun. 11, 2021, which in turn claims the benefit of Japanese Application No. 2021-025687, filed on Feb. 19, 2021, the entire disclosures of which Applications are incorporated by reference herein.

### TECHNICAL FIELD

The present invention relates to a sleep onset determination method, a sleep onset determination system, an air conditioning device, and a recording medium that determine sleep onset of a user.

### BACKGROUND ART

For example, Patent Literature (PTL) 1 discloses a sleep onset determination method. In this method, an illuminance level in a room is detected and sleep onset of a user is detected based on the illuminance level.

### CITATION LIST

### Patent Literature

[PTL 1] Japanese Unexamined Patent Application Publication No. 2012-202659

### SUMMARY OF INVENTION

### Technical Problem

In the above sleep onset determination method, when the illuminance level becomes less than or equal to a predetermined value, it is determined as sleep onset. However, in practice, the sleep onset cannot be detected accurately because of actions of a user going in or going out of the room in combination with operation of a light fixture.

The present invention aims to provide a sleep onset determination method, a sleep onset determination system, an air conditioning device, and a recording medium that are capable of determining sleep onset more highly accurately.

### Solution to Problem

A sleep onset determination method according to one aspect of the present invention includes: detecting a light-out time that is a time at which illuminance in a room becomes less than or equal to a predetermined value; storing the light-out time detected in the detecting of the light-out time and a time period including the light-out time detected; and determining whether a user fell asleep at a currently detected light-out time, based on the light-out time and the time period stored in the storing of the light-out time.

Furthermore, a sleep onset determination system according to one aspect of the present invention includes: an illuminance detection unit configured to detect a light-out time that is a time at which illuminance in a room becomes less than or equals to a predetermined value; a light-out time storage configured to store the light-out time detected by the illuminance detection unit and a time period including the light-out time detected; and a determination unit configured to determine whether a user fell asleep at a currently detected light-out time, based on the light-out time and the time period stored by the light-out time storage.

Furthermore, an air conditioning device according to one aspect of the present invention includes: a communication unit configured to obtain a determination result of the determination unit in the sleep onset determination system described above; an air conditioning unit configured to adjust a temperature in the room; and a control unit configured to control the air conditioning unit. The control unit is configured to control the air conditioning unit, based on the determination result of the determination unit obtained by the communication unit.

Furthermore, a recording medium according to one aspect of the present invention is a non-transitory computer-readable recording medium for use in a computer. The recording medium has a computer program recorded thereon for causing the computer to execute the above sleep onset determination method.

### Advantageous Effects of Invention

A sleep onset determination method and so on according to one aspect of the present invention makes it possible to determine sleep onset more highly accurately.

### BRIEF DESCRIPTION OF DRAWINGS

FIG. 1 is a block diagram illustrating a functional configuration of a sleep onset determination system according to an embodiment.

FIG. 2 is a flowchart of a learning process according to the embodiment.

FIG. 3 is a flowchart of a determination process according to the embodiment.

FIG. 4 is a block diagram illustrating a functional configuration of a sleep onset determination system according to a variation.

### DESCRIPTION OF EMBODIMENTS

The following describes, in detail, embodiments of a sleep onset determination method and so on according to the present invention, with reference to the drawings. Note that each of the embodiments described below shows a preferred specific example of the present invention. Therefore, the numerical values, shapes, materials, structural elements, the arrangement and connection of the structural elements, steps and the order of the steps mentioned in the following embodiments are mere examples and not intended to limit the present invention.

Note that the accompanying drawings and the following description are provided to help a person skilled in the art to fully understand the present invention, and are not intended to limit the subject matter defined in the appended claims.

Furthermore, the figures are schematic diagrams and are not necessarily precise illustrations. Throughout the figures, structural elements that are essentially the same share like reference signs. Accordingly, duplicate description is omitted or simplified.

### Embodiment

### Configuration

First, a configuration of a sleep onset determination system according to an embodiment will be described. Note that in the present embodiment, an example in which the sleep onset determination system is applied to an air conditioner (air conditioning device) will be described. Note that the sleep onset determination system may be applied to an electronic device other than an air conditioner. The electronic device other than an air conditioner may be any electronic device. It is sufficient as long as the electronic device can reflect sleep onset of a user on its control. Examples of such an electronic device include an automatic control device for a television, an air cleaner, a humidifier, a smart lock, a loudspeaker, a lighting fixture, or a curtain.

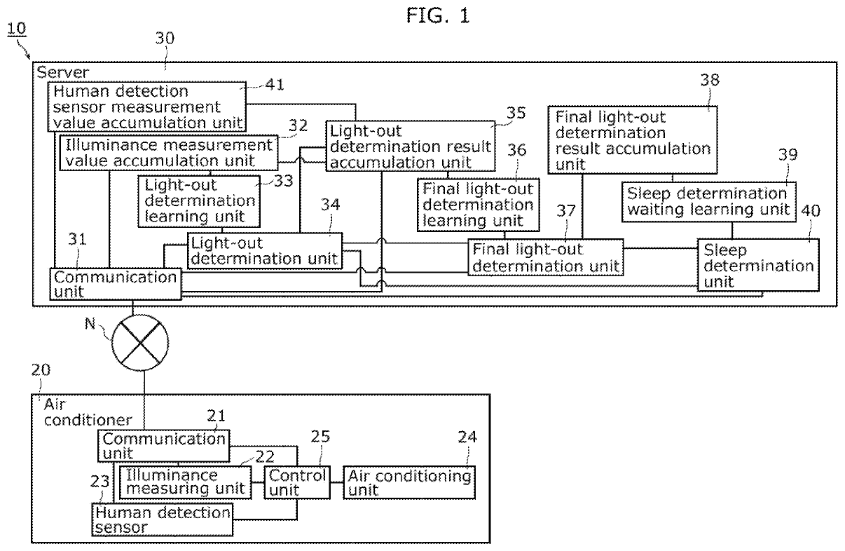

FIG. 1 is a block diagram illustrating a functional configuration of sleep onset determination system 10 according to the embodiment. As illustrated in FIG. 1, sleep onset determination system 10 includes air conditioner 20 and server 30. Air conditioner 20 and server 30 are connected to each other via network N.

### Air Conditioner

Air conditioner 20 is provided in a room of a home where a user lives, for example. Air conditioner 20 includes communication unit 21, illuminance measuring unit 22, human detection sensor 23, air conditioning unit 24, and control unit 25.

Communication unit 21 is a communication module connected to network N. Communication unit 21 is connected to illuminance measuring unit 22, human detection sensor 23, and control unit 25. Communication unit 21 connects server 30, illuminance measuring unit 22, human detection sensor 23, and control unit 25 such that these structural elements can freely communicate with each other via network N.

Illuminance measuring unit 22 is an illuminance sensor that detects illuminance in the room where air conditioner 20 is provided. Illuminance measuring unit 22 outputs an illuminance value measured at predetermined intervals (for example, at intervals of 5 minutes).

Human detection sensor 23 is a sensor that detects presence or absence of a user at a predetermined position in the room where air conditioner 20 is provided. Any device can be used as human detection sensor 23 as long as the device can detect a position of a user present in the room using any of the following: infrared rays, supersonic rays, and visible light. Human detection sensor 23 outputs user position information indicating presence or absence of the user detected at predetermined intervals (for example, at intervals of 5 minutes). Human detection sensor 23 may be a structural element different from air conditioner 20.

Air conditioning unit 24 is a drive unit for adjusting the temperature in the room. Control unit 25 is a microcomputer that controls air conditioning unit 24 based on a determination result input from server 30 via communication unit 21.

### Server

Server 30 is a server device (computer) connected to air conditioner 20 such that server 30 can freely communicate with air conditioner 20 via network N. Server 30 includes: communication unit 31, illuminance measurement value accumulation unit 32, human detection sensor measurement value accumulation unit 41, light-out determination learning unit 33, light-out determination unit 34, light-out determination result accumulation unit 35, final light-out determination learning unit 36, final light-out determination unit 37, final light-out determination result accumulation unit 38, sleep determination waiting learning unit 39, and sleep determination unit 40.

Note that each of the learning units (light-out determination learning unit 33, final light-out determination learning unit 36, and sleep determination waiting learning unit 39) outputs a learning model to a corresponding one of the determination units (light-out determination unit 34, final light-out determination unit 37, and sleep determination unit 40). Each determination unit gives an input value to its learning model and performs determination. The type of the learning model included in each learning unit may be any type of learning model without limitation. For example, machine learning may be used. A specific example of the learning model may be a neural network. Moreover, as a machine learning algorithm, for example, one of a decision tree, a support vector machine (SVM), k-nearest neighbors algorithm (kNN), a random forest, and gradient boosting may be used, or these may be used in any combination. Furthermore, it is preferable that each learning unit performs supervised learning, but each learning unit may perform unsupervised learning.

Communication unit 31 is a communication module connected to network N. Communication unit 31 is connected to illuminance measurement value accumulation unit 32, light-out determination unit 34, light-out determination result accumulation unit 35, final light-out determination unit 37, sleep determination unit 40, and human detection sensor measurement value accumulation unit 41. Communication unit 31 connects, via network N, to air conditioner 20, illuminance measurement value accumulation unit 32, light-out determination unit 34, light-out determination result accumulation unit 35, final light-out determination unit 37, sleep determination unit 40, and human detection sensor measurement value accumulation unit 41 such that illuminance measurement value accumulation unit 32, light-out determination unit 34, light-out determination result accumulation unit 35, final light-out determination unit 37, sleep determination unit 40, and human detection sensor measurement value accumulation unit 41 can freely communicate with air conditioner 20.

Illuminance measurement value accumulation unit 32 is a storage that accumulates, as a log, illuminance values of illuminance measuring unit 22. The illuminance values are each input from illuminance measuring unit 22 in air conditioner 20 via network N and communication unit 31.

Human detection sensor measurement value accumulation unit 41 is a storage that accumulates, as a log, detection results of human detection sensor 23. The detection results are each input from human detection sensor 23 in air conditioner 20 via network N and communication unit 31.

Light-out determination learning unit 33 learns whether the room where air conditioner 20 is provided is dark or bright by performing statistical processing based on the log of the illuminance values accumulated in illuminance measurement value accumulation unit 32. Specifically, light-out determination learning unit 33 determines an illuminance value when the room is dark (a first illuminance value). The illuminance value when the room is dark is an illuminance value measured over the longest time in a time frame that is generally considered to be a light-out time during the night (for example, AM 1:00-AM 5:00). Moreover, light-out determination learning unit 33 determines an illuminance value when the room is bright (a second illuminance value). The illuminance value when the room is bright is an illuminance value in a time frame that is generally considered to be a time before sleep onset during the night (for example, PM 8:00-AM 4:00), and is measured immediately after the first illuminance value. The illuminance value when the room is bright is also a value that is other than values in the lowest 10% of the accumulated illuminance values greater than the first illuminance value. The values in the lowest 10% of the accumulated values may be obtained in cases where the illuminance values do not result from turning on a light (for example, light enters the room from outside the room due to opening and closing the door). Therefore, excluding illuminance values in the lowest 10% of the accumulated illuminance values makes it possible to extract illuminance values resulting from turning on the light. Note that values in the lowest 10% of the accumulated illuminance values are used here, but any other values may be used.

Light-out determination unit 34 obtains an illuminance value currently measured by illuminance measuring unit 22 from communication unit 31, and determines a light-out time in the room based on the illuminance value and the learning model that has been learned by light-out determination learning unit 33. Specifically, light-out determination unit 34 determines, as a light-out time, a timing (time) at which the second illuminance value changes to the first illuminance value after the second illuminance value continues for at least a predetermined time (for example, 5 minutes to 10 minutes). In other words, in the present embodiment, light-out determination learning unit 33 and light-out determination unit 34 are an example of the illuminance detection unit that determines a light-out time at which the illuminance value in the room becomes less than or equal to a predetermined value (first illuminance value). Note that since the first illuminance value and the second illuminance value are determined using statistical processing in the present embodiment, the first illuminance value and the second illuminance value are determined according to an environment in which illuminance values are to be detected. However, the first illuminance value and the second illuminance value may be fixed values that are set in advance. In other words, in this case, the system configuration may be simplified because the light-out time can be detected without using statistical processing.

Light-out determination result accumulation unit 35 is a storage that associates, with one another, the light-out time determined by light-out determination unit 34, the time period including the light-out time detected, and user position information output from human detection sensor 23 at a time at which the light-out time is determined, and accumulates them as a log. Here, the time period includes a day of week, month, and season. Here, light-out determination result accumulation unit 35 is an example of a light-out time storage that stores a light-out time detected by illuminance detection unit and a time period including the light-out time detected.

Final light-out determination learning unit 36 learns whether light-out at a light-out time is the final light-out of a day through processing with its learning model, based on logs of the light-out times, time periods, and user position information that are accumulated in light-out determination result accumulation unit 35. For example, final light-out determination learning unit 36 obtains a determination result of each machine learning algorithm and its probability by using machine learning algorithms individually.

Note that it is difficult to determine in real time whether light-out at a light-out time is the final light-out of a day. However, checking one day data of a target day, which is a day for which the final light-out is to be determined, on the day following the target day makes it easier to determine whether the light-out at the light-out time is the final light-out of the target day. Final light-out determination learning unit 36 performs learning in view of the result determined on the day following the target day.

Final light-out determination unit 37 inputs the determination result of light-out determination unit 34 to the learning model obtained by final light-out determination learning unit 36 to determine whether the current light-out is the final light-out. Specifically, final light-out determination unit 37 extracts a determination result having the highest probability from among determination results and their probabilities determined by final light-out determination learning unit 36, and uses the extracted determination result as a result of final light-out determination. In other words, when final light-out determination unit 37 determines that the currently detected light-out time is the final light-out, final light-out determination unit 37 determines that the user in the room fell asleep. As described above, final light-out determination learning unit 36 and final light-out determination unit 37 are an example of a determination unit that determines whether the user fell asleep at the currently detected light-out time through processing a light-out time and its time period with a machine learning model. When final light-out determination unit 37 determines the final light-out, final light-out determination unit 37 outputs the determination result to air conditioner 20 from communication unit 31 via network N.

Final light-out determination result accumulation unit 38 is a storage that associates, the light-out time, the time period, and user position information that have been input to obtain the determination result adopted by final light-out determination unit 37 with the determination result and its probability, and stores them as a log.

After a predetermined time elapsed from when the determination has been performed by final light-out determination unit 37, sleep determination waiting learning unit 39 processes, using the learning model obtained by machine learning, (i) the probability of the determination result of final light-out determination unit 37; (ii) light-out times, time periods, and user position information that have been associated with the probability; and (iii) time elapsed after the light-out has been detected.

Specifically, sleep determination waiting learning unit 39 uses, as explanatory variables, parameters to be used in the above-described machine learning, and determines, as response variables, flags indicating whether light-out occurred again after the light-out time. For example, a flag is determined for each of the logs of illuminance measuring unit 22 transmitted at 5-minute intervals. When there is light-out after the light-out time, the flag is 0. When there is no light-out, the flag is 1. Sleep determination waiting learning unit 39 learns whether the flag is 0 or 1, based on the explanatory variables. Sleep determination waiting learning unit 39 learns the data by machine learning and creates a model that calculates a probability of whether the user fell asleep according to the time frame when the light-out has been detected and the time elapsed from the detection of light-out. Here, sleep determination waiting learning unit 39 uses a logistic regression model, for example.

For example, sleep determination waiting learning unit 39 learns that the probability that the user fell asleep at a current time increases as the time frame of the light-out time becomes later. Moreover, sleep determination waiting learning unit 39 learns that the probability that the user fell asleep at a current time increases as the probability of the determination for the light-out time immediately before the light-out time that is currently being determined increases. Moreover, sleep determination waiting learning unit 39 learns that the probability that the user fell asleep at a current time increases as the time elapsed from the time at which the light-out has been detected becomes longer.

Sleep determination unit 40 inputs, to the learning model obtained by sleep determination waiting learning unit 39, the determination result of light-out determination unit 34 and the probability of the determination result adopted by final light-out determination unit 37 to determine whether the user fell asleep at a current time. Specifically, sleep determination unit 40 calculates the probability of the determination using the learning result obtained by sleep determination waiting learning unit 39. Sleep determination unit 40 determines that the user fell asleep at a current time when the probability of the determination is greater than or equal to a predetermined value. As described above, sleep determination waiting learning unit 39 and sleep determination unit 40 are an example of a redetermination unit that redetermines whether the user fell asleep at the currently detected light-out time by processing a light-out time and its time period with a learning model after a predetermined time elapsed from the time at which determination by final light-out determination unit 37 has been performed. Then, sleep determination unit 40 outputs its determination result to air conditioner 20 from communication unit 31 via network N. Note that sleep determination unit 40 does not perform the determination when final light-out determination unit 37 determines the final light-out.

Each of the processing units of server 30 (illuminance measurement value accumulation unit 32, light-out determination learning unit 33, light-out determination unit 34, light-out determination result accumulation unit 35, final light-out determination learning unit 36, final light-out determination unit 37, final light-out determination result accumulation unit 38, sleep determination waiting learning unit 39, and sleep determination unit 40) may be implemented by a control program for executing the above-described processes and a central processing unit (CPU), a random-access memory (RAM), and a read-only memory (ROM) that execute the control program. Each of these processing units may be implemented by one or more CPUs.

### Sleep Onset Determination Method

Next, a sleep onset determination method to be executed by server 30 in sleep onset determination system 10 according to the present embodiment will be described. Note that the sleep onset determination method is stored as a program in server 30. The sleep onset determination method includes a learning process and a determination process.

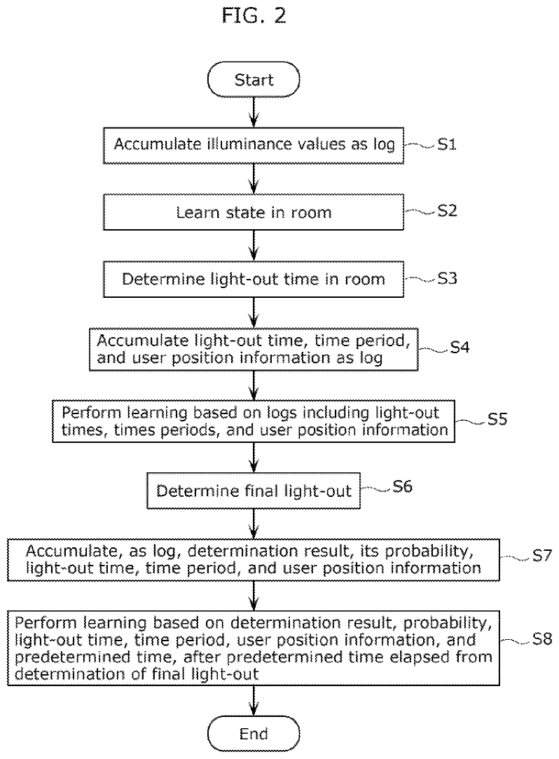

FIG. 2 is a flowchart of a learning process according to the embodiment. As illustrated in FIG. 2, in step S1, illuminance measurement value accumulation unit 32 accumulates, as a log, illuminance values of illuminance measuring unit 22. The illuminance values are input from illuminance measuring unit 22 of air conditioner 20 via network N and communication unit 31.

In step S2, light-out determination learning unit 33 learns whether the room where air conditioner 20 is provided is dark or bright by performing statistical processing based on the log of illuminance values accumulated in illuminance measurement value accumulation unit 32.

In step S3, light-out determination unit 34 determines, as a light-out time in the room, based on the contents learned by light-out determination learning unit 33, a timing (time) at which the second illuminance value changes to the first illuminance value after the second illuminance value continues for at least a predetermined time (for example, 5 minutes to 10 minutes). Accordingly, step S2 and step S3 are an example of detecting a light-out time that is a time at which illuminance in a room becomes less than or equals to a predetermined value.

In step S4, light-out determination result accumulation unit 35 associates, with one another, the light-out time determined by light-out determination unit 34, the time period including the light-out time detected, and user position information output from human detection sensor 23 at a time at which the light-out time is determined, and accumulates them as a log. Accordingly, step S4 is an example of storing the light-out time detected in the detecting of the light-out time and a time period including the light-out time detected. Furthermore, step S4 is also an example of detecting presence or absence of the user at a predetermined position in the room, as well as an example of storing user position information indicating the presence or absence of the user detected in the detecting of presence or absence of the user. The present embodiment describes an example in which the storing of the light-out time, the detecting of presence or absence of the user, and the storing of user position information are executed in step S4. However, these may be executed independently of each other.

In step S5, final light-out determination learning unit 36 learns, through processing with its learning model, whether the light-out at a current light-out time is the final light-out of the day, based on the logs including the light-out times, their time periods, and user position information accumulated in light-out determination result accumulation unit 35.

In step S6, final light-out determination unit 37 extracts a determination result having the highest probability from among the determination results and their probabilities determined by final light-out determination learning unit 36, and adopts the extracted determination result as a result of final light-out determination. Accordingly, step S5 and step S6 are an example of determining whether the user fell asleep at the currently detected light-out time through processing, using a learning model, (i) the light-out time and the time period stored in the storing of the light-out time, and (ii) the user position information stored in the storing of the user position information.

In step S7, final light-out determination result accumulation unit 38 associates, the light-out time, the time period, and user position information that have been input to obtain the determination result, with the determination result adopted by final light-out determination unit 37 and the probability of the determination result, and stores accumulates as a log. Accordingly, step S7 is an example of storing a determination result of the determining.

In step S8, sleep determination waiting learning unit 39 learns, through processing, using its learning model, (i) the probability of the determination result of final light-out determination unit 37; (ii) the light-out time, time period, and user position information that have been associated with the probability; and (iii) time elapsed after the light-out has been detected. This processing is performed after a predetermined time elapsed from when the determination by final light-out determination unit 37 has been performed. Then, sleep determination waiting learning unit 39 ends the learning process.

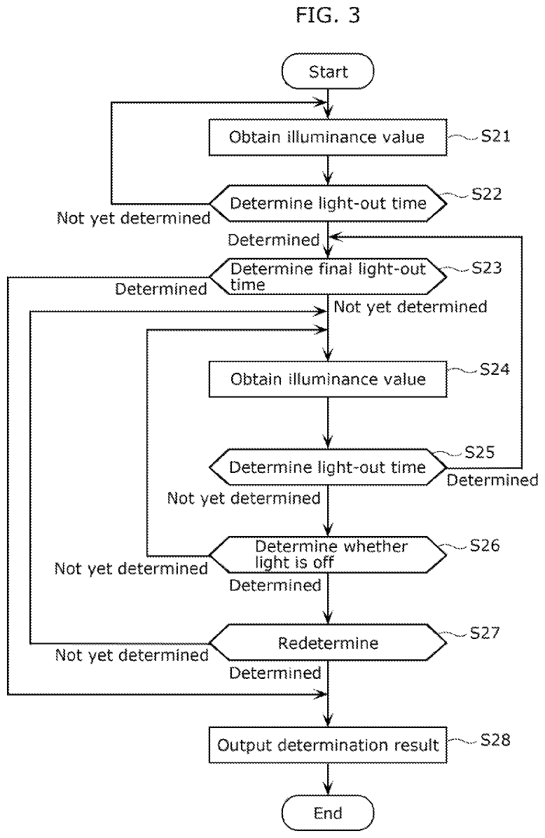

Next, a determination process will be described. FIG. 3 is a flowchart of a determination process according to the embodiment. As illustrated in FIG. 3, in step S21, light-out determination unit 34 obtains an illuminance value of illuminance measuring unit 22. The illuminance value is input from illuminance measuring unit 22 of air conditioner 20 via network N and communication unit 31.

In step S22, light-out determination unit 34 obtains the illuminance value currently measured by illuminance measuring unit 22 from communication unit 31, and determines a light-out time in the room based on the illuminance value and the learning model obtained by light-out determination learning unit 33. Here, light-out determination unit 34 proceeds to step S21 when the light-out time is not yet determined and proceeds to step S23 when the light-out time is determined.

In step S23, final light-out determination unit 37 inputs the determination result of light-out determination unit 34 to the learning model obtained by final light-out determination learning unit 36 to determine whether the current light-out is the final light-out. Step S23 is an example of determining whether a user fell asleep at a currently detected light-out time. Here, final light-out determination unit 37 proceeds to step S24 when the final light-out time is not yet determined and proceeds to step S28 when the final light-out time is determined.

In step S24, light-out determination unit 34 obtains an illuminance value of illuminance measuring unit 22. The illuminance value is input from illuminance measuring unit 22 in air conditioner 20 via network N and communication unit 31.

In step S25, light-out determination unit 34 obtains the illuminance value currently measured by illuminance measuring unit 22 from communication unit 31, and determines a light-out time in the room based on the illuminance value and the learning model obtained by light-out determination learning unit 33. Here, light-out determination unit 34 proceeds to step S26 when the light-out time is not yet determined and proceeds to step S23 when the light-out time is determined.

In step S26, light-out determination unit 34 determines whether the light in the room is off, based on the illuminance value obtained in step S25. Here, light-out determination unit 34 proceeds to step S24 when it is not yet determined that the light in the room is off and proceeds to step S27 when it is determined that the light in the room is off.

In step S27, sleep determination unit 40 inputs, to the learning model obtained by sleep determination waiting learning unit 39, the determination result of light-out determination unit 34 and the probability of the determination result adopted by final light-out determination unit 37 to determine whether the user fell asleep at a current time. As described above, step S8 and step S27 are an example of redetermining whether the user fell asleep after a predetermined time elapsed from the currently detected light-out time. This redetermining is performed by processing, using the learning model, the light-out time and the time period stored in the storing after a predetermined time elapsed from when the determining has been performed. Here, sleep determination unit 40 proceeds to step S24 when it is not yet determined that the user fell asleep, and proceeds to step S28 when it is determined that the user fell asleep.

In step S28, final light-out determination unit 37 or sleep determination unit 40 transmits its determination result to air conditioner 20 from communication unit 31 via network N. In other words, step S28 is an example of transmitting a control signal to a device (air conditioner 20) in the room, when it is determined that the user fell asleep in the determining. In air conditioner 20, when communication unit 21 receives the determination result of final light-out determination unit 37 or sleep determination unit 40, control unit 25 controls air conditioning unit 24 based on the determination result. In other words, after the light-out time is determined to be a time at which the user fell asleep, the temperature in the room will be controlled in a mode suitable for sleeping.

### Effects Etc.

As described above, the sleep onset determination method according to the embodiment includes: detecting a light-out time that is a time at which illuminance in a room becomes less than or equal to a predetermined value; storing the light-out time detected in the detecting of the light-out time and a time period including the light-out time detected; and determining whether a user fell asleep at a currently detected light-out time, based on the light-out time and the time period stored in the storing of the light-out time.

Moreover, the program according to the embodiment is a program for causing a computer to execute the sleep onset determination method described above.

Moreover, sleep onset determination system 10 according to the embodiment includes: an illuminance detection unit (light-out determination learning unit 33 and light-out determination unit 34) configured to detect a light-out time that is a time at which illuminance in a room becomes less than or equals to a predetermined value; a light-out time storage (light-out determination result accumulation unit 35) configured to store the light-out time detected by the illuminance detection unit and a time period including the light-out time detected; and a determination unit (final light-out determination learning unit 36 and final light-out determination unit 37) configured to determine whether a user fell asleep at a currently detected light-out time, based on the light-out time and the time period stored by the light-out time storage.

Here, the inventors have studied a case where the light-out time detected in the step of detecting a light-out time is determined to be a timing that a user fell asleep (sleep onset timing). In this case, an error occurred at a rate of approximately 25% that it is determined that the user fell asleep at a timing earlier than the actual final light-out time of the day (the timing at which the user actually fell asleep) in the determination.

In the present embodiment, it is determined that the user fell asleep at the currently detected light-out time by detecting light-out times when the illuminance in the room becomes less than or equal to a predetermined value and then processing the light-out times and their time periods with a learning model. With this, considering the past tendency makes it possible to determine whether the currently detected light-out time is the final light-out of a day, i.e., a timing at which the user fell asleep. In this case, the above-described rate of error in the determination was reduced to approximately 10%, according to the studies by the inventors. Accordingly, sleep onset can be determined more highly accurately.

In particular, a sleep onset timing of a user is determined by considering the time period including the light-out time detected. Examples of the time period include a day of week, month, and season, as described above. The daily habit of a user tends to differ depending on such time periods. For example, a user who works differently depending on days of a week, there may be a certain tendency in a timing of falling asleep for each day of a week. Moreover, there may be a user who falls asleep later in summer and earlier in winter. When a sleep onset timing is determined by considering such time periods, the sleep onset timing can be determined more accurately for a user who changes his/her daily habit depending on the time periods. Note that statistical processing may be performed using data having a high similarity in characteristics of time periods and the processed data may be reflected on sleep onset timing determination.

Moreover, the air conditioner (air conditioning device) according to the embodiment includes: communication unit 21 that obtains a determination result of the determination unit in sleep onset determination system 10 described above; air conditioning unit 24 configured to adjust a temperature in the room; and control unit 25 configured to control air conditioning unit 24. Control unit 25 is configured to control air conditioning unit 24, based on the determination result of the determination unit obtained by communication unit 21.

Since this makes it possible to reflect more highly accurate sleep onset determination on a control by air conditioning unit 24, a mode suitable for sleep can be performed more reliably after the user fell asleep.

Moreover, the determining includes determining whether the user fell asleep at the currently detected light-out time through processing, using a learning model, the light-out time and the time period stored in the storing of the light-out time.

With this, the light-out time and the time period stored in the storing of the light-out time are processed using a learning model to determine that the user fell asleep at the currently detected light-out time. Therefore, sleep onset of a user can be determined more accurately.

Moreover, the sleep onset determination method includes: storing a determination result of the determining; and redetermining whether the user fell asleep after a predetermined time elapsed from the currently detected light-out time, through processing the light-out time and the time period stored in the storing of the light-out time, the processing being performed using a learning model obtained by machine learning after the predetermined time elapsed from when the determining has been performed.

For example, when the determination result obtained in the determining is simply adopted, i.e., when the redetermining is not performed, an error may occur that it is continuously determined that a currently detected light-out time is not the final light-out time in a day, and a final determination result is not determined. According to studies by the inventors, such an error occurs at a rate of approximately 8% when the redetermining is not performed.

In the present embodiment, it is redetermined whether the user fell asleep at the currently detected light-out time by processing, using a learning model obtained by machine learning, a log including light-out times and time periods after a predetermined time elapsed from when the determining has been performed. It is possible to determine whether the currently detected light-out time is the final light-out of a day, i.e., a timing at which the user fell asleep, by considering the past tendency after the predetermined time elapsed from when the determining has been performed. In this case, the rate of error described above is reduced to approximately 4% according to the studies by the inventors. Accordingly, sleep onset can be determined more highly accurately.

Furthermore, the sleep onset determination method includes detecting presence or absence of the user at a predetermined position in the room; and storing user position information indicating the presence or absence of the user that has been detected in the detecting of the presence or absence of the user. The determining includes determining whether the user fell asleep at the currently detected light-out time, through processing, using a learning model obtained by machine learning, the user position information stored in the storing of the user position information, in addition to the light-out time and the time period stored in the storing of the light-out time.

With this, in the determining, determination is performed including the user position information. In other words, sleep onset determination can be determined more highly accurately, because it is determined that a user fell asleep at a currently detected light-out time by considering the position of the user in a room. For example, it is possible to reduce cases where the light-out time at which a user turned the light off when leaning the room is erroneously determined to the sleep onset timing.

Moreover, the sleep onset determination method further includes transmitting a control signal to a device (air conditioner 20) in the room when it is determined that the user fell asleep in the determining.

With this, the determination result that the user fell asleep can be reflected on a control of a device in the room. Therefore, it is possible to cause a device to perform a mode suitable for sleep after the user fell asleep.

### Other Embodiments

Hereinbefore, one or more aspects of the sleep onset determination system and so on according to the present invention have been described based on the embodiment, but the present invention should not be limited to the embodiment.

For example, in the embodiment, an example has been described in which the sleep onset determination method includes detecting presence or absence of the user and storing user position information. However, the sleep onset determination method does not need to include the detecting of presence or absence of the user and the storing of user position information.

Moreover, in the embodiment, an example has been described in which a sleep onset determination method includes storing a determination result and redetermining. However, the sleep onset determination method does not need to include the storing of a determination result and the redetermining.

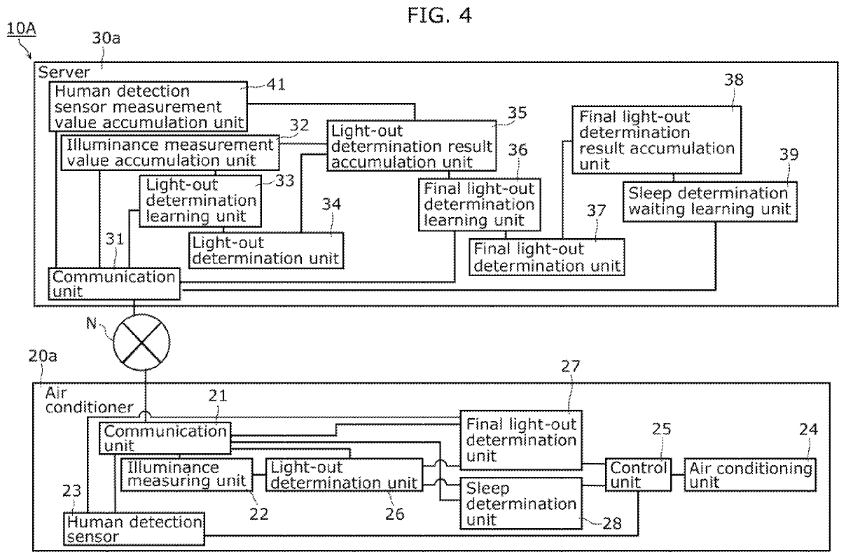

Moreover, in the embodiment, an example has been described in which server 30 includes: illuminance measurement value accumulation unit 32, light-out determination learning unit 33, light-out determination unit 34, light-out determination result accumulation unit 35, final light-out determination learning unit 36, final light-out determination unit 37, final light-out determination result accumulation unit 38, sleep determination waiting learning unit 39, sleep determination unit 40, human detection sensor measurement value accumulation unit 41. However, these structural elements may be provided only in the air conditioner, or provided in the server and the air conditioner separately. For example, FIG. 4 illustrates an example in which these structural elements are provided in the server and the air conditioner separately.

FIG. 4 is a block diagram illustrating a functional configuration of sleep onset determination system 10A according to a variation. Note that in the following description, structural elements substantially the same as the structural elements in the embodiment are assigned the same reference signs and description thereof may be omitted.

As illustrated in FIG. 4, when server 30a is compared with the server in the embodiment, the sleep determination unit is removed from server 30a. Final light-out determination learning unit 36 and sleep determination waiting learning unit 39 can freely communicate with communication unit 21 in air conditioner 20a via communication unit 31 and network N. Air conditioner 20a includes light-out determination unit 26, final light-out determination unit 27, and sleep determination unit 28. Each determination is performed by receiving a learning model from light-out determination unit 33, final light-out determination learning unit 36, and sleep determination waiting learning unit 39, via communication unit 21, network N, and communication unit 31. Moreover, human detection sensor 23 is connected to final light-out determination unit 27 such that human detection sensor 23 can freely communicate with final light-out determination unit 27. The sleep onset determination system according to the present invention can be implemented also when the learning units and the determination units are provided in separate devices as in this variation. Specifically, in the present variation, since light-out determination unit 26 obtains a learning model from light-out determination learning unit 33, light-out determination unit 26 may obtain a current illuminance value from illuminance measuring unit 22. In other words, light-out determination unit 36 does not need to obtain a log from illuminance measurement value accumulation unit 32, it is possible to reduce a communication load between server 30a and determination unit 36.

Moreover, in the above embodiment, it has been described that each of the processing units of server 30 (illuminance measurement value accumulation unit 32, light-out determination learning unit 33, light-out determination unit 34, light-out determination result accumulation unit 35, final light-out determination learning unit 36, final light-out determination unit 37, final light-out determination result accumulation unit 38, sleep determination waiting learning unit 39, sleep determination unit 40) are implemented by a CPU and a control program. For example, the structural elements of each of the processing units may be configured by one or more electronic circuits. The one or more electronic circuits may be each a general-purpose circuit or a dedicated circuit. The one or more electronic circuits may include, for example, a semiconductor device, an integrated circuit (IC), or a large scale integration (LSI). The IC or LSI may be integrated into a single chip or multiple chips. Due to a difference in the degree of integration, the electronic circuit referred here to as an IC or LSI may be referred to as a system LSI, very large scale integration (VLSI), or ultra large scale integration (ULSI). Furthermore, a field programmable gate array (FPGA) which programmable after manufacturing of the LSI can be used for the same purposes.

Furthermore, the general and specific aspects of the present invention may be implemented using a system, a device, a method, an integrated circuit, or a computer program. Alternatively, these aspects may be implemented using a non-transitory computer-readable recording medium such as an optical disk, hard disk drive (HDD), or semiconductor memory storing the computer program. Furthermore, these aspects may be implemented using any combination of systems, devices, methods, integrated circuits, computer programs, or recording media.

Other than the above, the present invention also includes embodiments as a result of adding various modifications that may be conceived by those skilled in the art to the embodiment, and embodiments obtained by combining structural elements and functions in the embodiment in any manner as long as the combination does not depart from the scope of the present invention.

### INDUSTRIAL APPLICABILITY

The present invention is applicable to an electronic device that is capable of reflecting sleep onset of a user on its control.

### REFERENCE SIGNS LIST

10, 10A sleep onset determination system

20, 20a air conditioner

21, 31 communication unit

22 illuminance measuring unit

23 human detection sensor

24 air conditioning unit

25 control unit

26, 34 light-out determination unit (illuminance detection unit)

27, 37 final light-out determination unit (determination unit)

28, 40 sleep determination unit

30, 30a server

32 illuminance measurement value accumulation unit

33 light-out determination learning unit (illuminance detection unit)

35 light-out determination result accumulation unit (light-out time storage)

36 final light-out determination learning unit (determination unit)

38 final light-out determination result accumulation unit

39 sleep determination waiting learning unit

41 human detection sensor measurement value accumulation unit

N network

1. A sleep onset determination method comprising:
detecting a light-out time that is a time at which illuminance in a room becomes less than or equal to a predetermined value;
storing the light-out time detected in the detecting of the light-out time and a time period including the light-out time detected; and
determining whether a user fell asleep at a currently detected light-out time, based on the light-out time and the time period stored in the storing of the light-out time.

2. The sleep onset determination method according to claim 1,
wherein the determining includes determining whether the user fell asleep at the currently detected light-out time through processing, using a learning model, the light-out time and the time period stored in the storing of the light-out time.

3. The sleep onset determination method according to claim 1, further comprising:
storing a determination result of the determining; and
redetermining whether the user fell asleep after a predetermined time elapsed from the currently detected light-out time, through processing the light-out time and the time period stored in the storing of the light-out time, the processing being performed using a learning model obtained by machine learning after the predetermined time elapsed from when the determining has been performed.

4. The The sleep onset determination method according to claim 1, further comprising:
detecting presence or absence of the user at a predetermined position in the room; and
storing user position information indicating the presence or absence of the user that has been detected in the detecting of the presence or absence of the user,
wherein the determining includes determining whether the user fell asleep at the currently detected light-out time, through processing, using a learning model obtained by machine learning, the user position information stored in the storing of the user position information, in addition to the light-out time and the time period stored in the storing of the light-out time.

5. The sleep onset determination method according to claim 1, further including:
transmitting a control signal to a device in the room when it is determined that the user fell asleep in the determining.

6. A sleep onset determination system comprising:
an illuminance detection unit configured to detect a light-out time that is a time at which illuminance in a room becomes less than or equals to a predetermined value;
a light-out time storage configured to store the light-out time detected by the illuminance detection unit and a time period including the light-out time detected; and
a determination unit configured to determine whether a user fell asleep at a currently detected light-out time, based on the light-out time and the time period stored by the light-out time storage.

7. An air conditioning device comprising:
a communication unit configured to obtain a determination result of the determination unit in the sleep onset determination system according to claim 6;
an air conditioning unit configured to adjust a temperature in the room; and
a control unit configured to control the air conditioning unit,
wherein the control unit is configured to control the air conditioning unit, based on the determination result of the determination unit obtained by the communication unit.

8. A non-transitory computer-readable recording medium for use in a computer, the recording medium having a computer program recorded thereon for causing the computer to execute the sleep onset determination method according to claim 1.
